(19) United States
(12) Patent Application Publication
(10) Pub. No.: US 2008/0065205 A1
(43) Pub. Date: Mar. 13, 2008

(54) Retrievable implant and method for treatment of mitral regurgitation

(76) Inventors: Duy Nguyen, Corona, CA (US); Kim Nguyen, Irvine, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/519,519

(22) Filed: Sep. 11, 2006

(51) Int. Cl. A61F 2/24

(52) U.S. Cl. 623/2.36

(57) ABSTRACT

The invention is a device, and method for deploying same, configured for placement in a body lumen such as a coronary sinus, as may be desired to repair a mitral valve. The device includes a first anchor, a second anchor, and a bridge. The anchors are configured to delivered to a desired deployment site within the body lumen in a collapsed or contracted condition, and then be deployed by expanding the anchors into contact with the walls of the body lumen. One or more of the anchors may be configured to be radially collapsible after initial deployment in order to permit the anchor(s) to be repositioned in or removed from the body lumen. An anchor may be collapsible in response to a distal force applied to a portion of a proximal end thereof. A catheter for use with the implant is configured to deliver the implant to the site with the anchors in their respective collapsed configurations, and to release the anchors to permit them to expand into contact with the walls of the body lumen. The catheter is configured to apply a proximal force to one or both anchors, which may be applied via a cinch wire. The catheter may also be configured to apply a distal force against a portion of the proximal end of one or more of the anchors.

### FIELD OF THE INVENTION

The present invention relates to an implant to treat a deficient mitral valve, and more specifically to a retractable and/or retrievable implant to reduce mitral regurgitation.

### BACKGROUND OF THE INVENTION

Heart valve regurgitation, or leakage from the outflow to the inflow side of a heart valve, is a condition that occurs when a heart valve fails to close properly. Regurgitation through the mitral valve is often caused by changes in the geometric configurations of the left ventricle, papillary muscles, and mitral annulus. Similarly, regurgitation through the tricuspid valve is often caused by changes in the geometric configurations of the right ventricle, papillary muscles, and tricuspid annulus. These geometric alterations can result in incomplete coaptation of the valve leaflets during systole.

A variety of heart valve repair procedures have been proposed over the years for treating defective heart valves. With the use of current surgical techniques, it has been found that many regurgitant heart valves can be repaired.

In recent years, several new minimally invasive techniques have been introduced for repairing defective heart valves wherein open surgery and cardiopulmonary by-pass are not required. Some of these techniques involve introducing an implant into the coronary sinus for remodeling the mitral annulus. The coronary sinus is a blood vessel that extends around a portion of the heart through the atrioventricular groove in close proximity to the posterior, lateral, and medial aspects of the mitral annulus. Because of its position, the coronary sinus provides an ideal conduit for receiving an implant (i.e., endovascular device) configured to act on the mitral annulus. Examples of mitral valve repair devices insertable into the coronary sinus are described in U.S. patent application Ser. No. 11/014,273, filed Dec. 15, 2004, the entire contents of which are incorporated herein by reference.

When mitral valve repair devices are inserted into a patient, there may be a need to reposition the device after the anchors have been secured if the initial location of the device is not ideal. Thus, there is a need for a mitral valve repair that is easily retrievable once it has been deployed in a patient. More specifically, there is a need for a mitral valve repair device and system having anchors that can be easily retracted after initial deployment and then repositioned. The current invention fulfills this need.

### SUMMARY OF THE INVENTION

Preferred embodiments of the present invention provide an implant, and method of use therefore, configured for placement in a body lumen such as the coronary sinus. The implant has a first anchor, a second anchor, and a connecting bridge that connects the first anchor to the second anchor. The first and second anchors are configured to radially expand into contact with the walls of the body lumen so that the anchors are secured within the body lumen. The first and/or second anchors are configured to be retrievable after deployment. For example, an anchor may be radially collapsible after deployment, with the anchor configured to radially collapse in response to the application of a generally longitudinal force applied to the anchor. The longitudinal force may be a distally-directed force applied against a portion of a proximal end of the anchor.

The first and/or second anchor may be self-expanding, and may be formed from a memory material such as nitinol. The first and/or second anchors may be formed from a plurality of wire-like elements. In the expanded condition, the first and/or second anchors may each include a generally open proximal end, a generally open distal end, and a generally open central lumen. The first and/or second anchors may each include a wire mesh-like structure over an otherwise open distal end or an otherwise open proximal end.

An anchor according to the invention may have a generally tapering proximal end. The proximal end may be generally dome-shaped, or may be generally wedge-shaped. The distal end of an anchor according to the invention may be generally flared. An anchor may be formed by one or more generally helical coils. A first helical coil of a particular anchor may coil in a first direction, while a second helical coil of the same anchor may coil in a second direction opposite to the first direction. The anchor may include a covering over one or more of the helical coils.

The connecting bridge may be configured to selectively vary in length. The bridge may comprise a spring-like structure and a bioresorbable material, and may be configured to vary its length as the bioresorbable material is absorbed into the body.

The bridge may also or alternatively be slidingly disposed with respect to one or more of the anchors, so that one or more of the anchors can be slidingly advanced along the material forming the bridge toward or away from the opposing anchor. The bridge length can thus be varied by sliding the bridge with respect to one or more of the anchor. The implant may include a lock that prevents sliding of the bridge with respect to an anchor in one or more directions.

The invention can include a delivery catheter configured to receive the implant therein. The delivery catheter may include an inner member and an outer sheath slidingly disposed about the inner member. The inner member may be configured to receive a collapsed implant thereon, with the outer sheath configured to slide over the collapsed implant and retain the implant in the collapsed configuration. The delivery catheter may be configured to apply a proximal force to an anchor or other part of an implant, such as by pulling on the implant via a cinch wire or other element attached to the implant. The outer sheath may include a distal opening configured to receive a collapsed/contracted anchor or implant therein. The outer sheath may also include a distal edge configured to be engaged against a portion of an anchor proximal end, such as a tapering proximal end, to thereby cause the anchor to collapse to its contracted configuration. The delivery catheter may include a gripping element configured to grasp a portion of the implant, a cinch wire, a guide wire, or other items.

Other objects, features, and advantages of the present invention will become apparent from a consideration of the following detailed description.

### BRIEF DESCRIPTION OF THE DRAWINGS

FIG. 1 is a perspective view of an implant deployed within a coronary sinus according to an embodiment of the invention;

FIG. 2A is a side view of an exemplary implant having distal and proximal anchors, with the implant in the delivery configuration, according to an embodiment of the invention;

FIGS. 2B and 2C are end views of the distal anchor and the proximal anchor, respectively, of the implant of FIG. 2A;

FIG. 3A is a side view of the implant of FIG. 2A, with the implant in the deployed configuration;

FIGS. 3B and 3C are end views of the distal anchor and the proximal anchor, respectively, of the implant of FIG. 3A;

FIGS. 4A-4E are schematic side views in partial cross section of a delivery system deploying an implant within a body lumen;

FIGS. 4F-4G are schematic side views in partial cross section of a delivery system retrieving an implant within a body lumen;

FIG. 5A is a side view of an anchor according to an embodiment of the present invention;

FIGS. 5B and 5C are distal and proximal end views, respectively, of the anchor of FIG. 5A;

FIG. 6A is a side view of an anchor in a delivery configuration according to an embodiment of the present invention;

FIGS. 6B and 6C are top and proximal end views, respectively, of the anchor of FIG. 6A in a delivery configuration;

FIGS. 6D, 6E, and 6F are side, top, and proximal end views, respectively, of the anchor of FIG. 6A in an expanded configuration;

FIG. 7A is a perspective view of an implant in a deployed/expanded configuration according to an embodiment of the invention;

FIG. 7B is a side view of the implant of FIG. 7A;

FIGS. 7C and 7D are end views of the distal anchor and the proximal anchor, respectively, of the implant of FIG. 7A;

FIGS. 7E and 7F are perspective and side views, respectively, of the implant of FIG. 7A in a delivery configuration;

FIG. 8A is a perspective view of another embodiment of an implant of the present invention;

FIG. 8B is a side view of the implant of FIG. 8A;

FIG. 8C is an end view of the distal anchor of the implant of FIGS. 8A and 8B;

FIG. 9 is a perspective view of a distal anchor covered by a sleeve according to an embodiment of the current invention;

FIG. 10A is a perspective view of an embodiment of an implant of the present invention;

FIG. 10B is a side view of the implant of FIG. 10A;

FIG. 10C is an end view of the distal anchor of the implant of FIGS. 10A and 10B;

FIG. 11 is a top view of a bridge for use with an implant according to an embodiment of the present invention;

FIG. 12 is a side view of an implant according to an embodiment of the invention; and

FIG. 13 is a side view of an implant according to an embodiment of the invention.

### DETAILED DESCRIPTION OF PREFERRED EMBODIMENTS

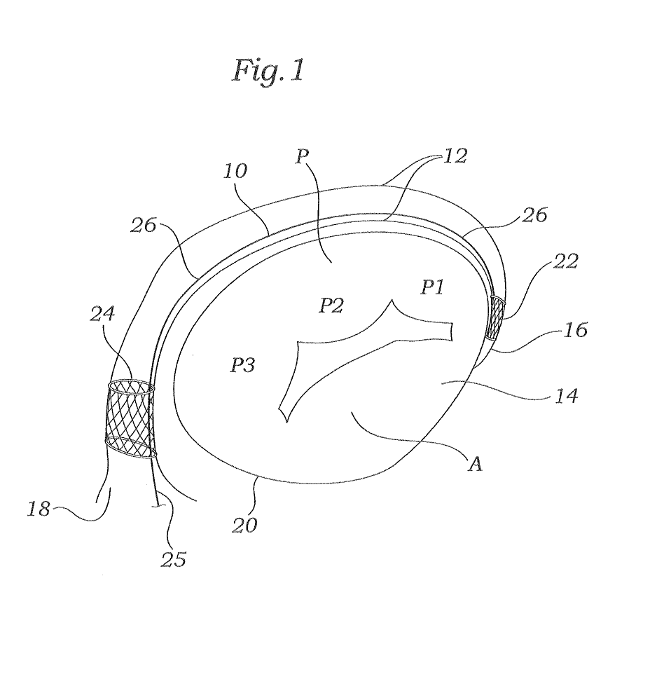

FIG. 1 depicts an implant 10 of the current invention deployed in the coronary sinus 12 of a mitral valve 14. From this view, it can be seen that the coronary sinus 12 extends around a posterior region of the mitral valve 14. The coronary sinus 12 is a relatively large vessel that receives venous drainage from the heart muscle. Blood flows through the coronary sinus 12 from a relatively narrow distal portion 16 and empties into the right atrium through a relatively wide coronary ostium 18. The mitral valve 14 generally includes an anterior leaflet A and a posterior leaflet P. The posterior leaflet P is formed with three scallops P1, P2, and P3. A mitral valve annulus 20 is a portion of tissue surrounding the mitral valve 14 to which the valve leaflets A, P attach. The coronary sinus 12 passes around the mitral valve 14 generally parallel to the mitral valve annulus 20 adjacent the posterior leaflet P.

As used herein, the term coronary sinus 12 is used as a generic term that describes the portion of the vena return system that is primarily situated adjacent to the mitral valve 14 and extends, at least in part, along the atrioventricular groove. Accordingly, the term “coronary sinus” may be construed to include the great cardiac vein and all other related portions of the vena return system.

It has been found that dilation of the mitral valve annulus 20 is the primary cause of regurgitation (i.e., reversal of flow) through the mitral valve 14. More particularly, when a posterior aspect (i.e., portion adjacent the posterior leaflet P) of the mitral valve annulus 20 dilates, one or more of the posterior leaflet scallops P1, P2, or P3 typically moves away from the anterior leaflet P. As a result, the anterior and posterior leaflets A, P fail to properly align and meet to completely close the mitral valve 14, and blood is capable of flowing backward through the resulting gap.

Reducing the dilation of the posterior aspect of the mitral valve annulus 20 can reduce and even eliminate mitral regurgitation. It has been found that applying tension within the coronary sinus 12 can alter the curvature of the coronary sinus 12, and thereby create a corresponding change in the dilation of the posterior aspect of the mitral valve annulus 20. As depicted in FIG. 1, the implant 10 applies tension within the coronary sinus 12, thereby pulling the coronary sinus 12 into a more straightened (i.e., less curved or dilated) configuration, which creates a corresponding reshaping of the posterior aspect of the mitral valve annulus 20. The implant 10 thus causes movement the posterior aspect of the mitral valve annulus 20 in an anterior direction, thereby moving the posterior leaflet P closer to the anterior leaflet A and closing the gap caused by the leaflet displacement.

The implant 10 includes a distal anchor 22, a proximal anchor 24, and a connecting bridge 26. The distal anchor 22 is depicted deployed in a generally narrow portion of the coronary sinus 12, while the proximal anchor 24 is deployed in a somewhat wider portion of the coronary sinus 12 adjacent the coronary ostium 18. The connecting bridge 26 pulls the distal and proximal anchors 22, 24 toward each other, thereby changing the curvature of the coronary sinus 12 and moving the posterior leaflet P toward the anterior leaflet A.

As used herein, “distal” means the direction of a device as it is being inserted into a patient's body or a point of reference closer to the leading end of the device as it is inserted into a patient's body. Similarly, as used herein “proximal” means the direction of a device as it is being removed from a patient's body or a point of reference closer to a trailing end of the device as it is inserted into a patient's body.

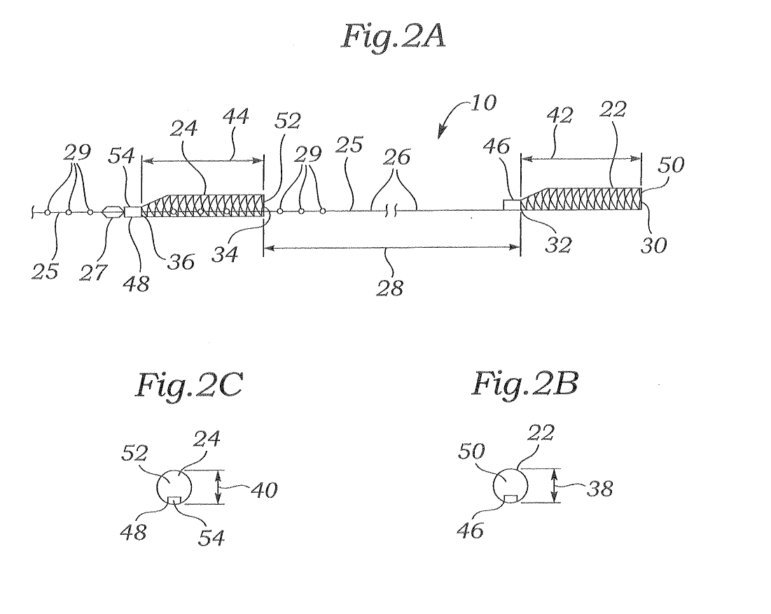

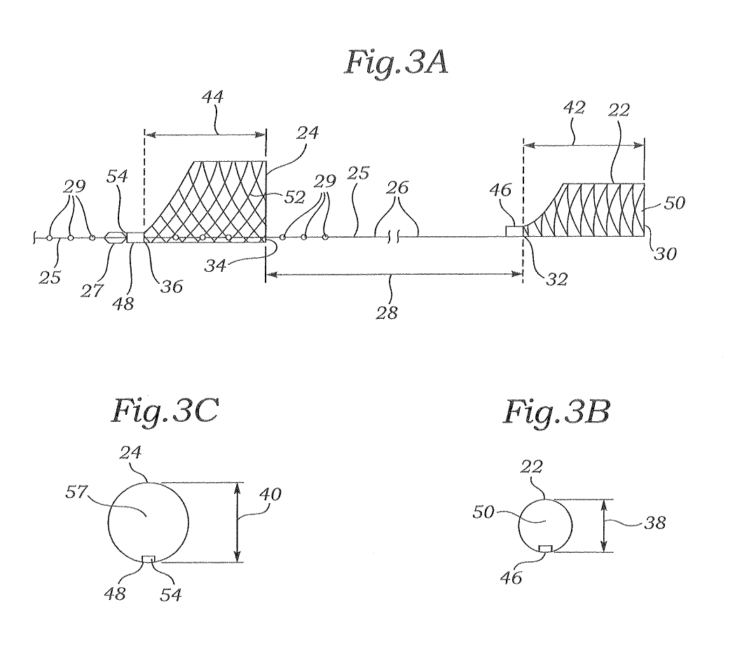

The implant 10 of FIG. 1 is depicted in greater detail in FIGS. 2A-2C (delivery configuration) and FIGS. 3A-3C (deployed/use configuration). FIGS. 2A and 3A depict the implant 10 viewed from the side. FIGS. 2B and 3B depict an end view of the distal anchor 22 looking distally along the distal anchor 22. FIGS. 2C and 3C depict an end view of the proximal anchor 24 looking distally along the proximal anchor 24. In the particular embodiment, the distal anchor 22 and proximal anchor 24 are formed from biocompatible mesh wire, such as nitinol or stainless steel wire. The distal anchor 22 and/or proximal anchor 24 may be formed from a shape memory material such as Nitinol to be self-expandable and biased into their use/deployed configuration.

The bridge 26 separates the distal and proximal anchors 22, 24. The bridge 26 has a length 28, defined as the length of bridge 26 extending between the distal and proximal anchors 22, 24. Depending on the particular embodiment, the bridge 26 may be adapted to selectively vary its length 28. For example, the bridge 26 may be configured to reduce its length 28 via the use of memory metals, resorbable materials, etc. For example, the bridge may be adapted to be threaded with a resorbable material, such as a coil or X-shape bridge structure threaded with resorbable thread. Resorbable materials are those that, when implanted into a human or other animal body, are resorbed by the body by means of enzymatic degradation and/or by the active absorption by blood and tissue cells of the body. The bridge 26 may also or alternatively be slidingly disposed with respect to one or more of the anchors 22, 24, so that one or more of the anchors 22, 24 can be slidingly advanced along the material forming the bridge 26 toward or away from the opposing anchor. These and other bridges having various configurations as are generally known in the art are within the scope of the invention.

In the particular embodiment of FIGS. 2A and 3A, the bridge 26 is formed by a cinching wire 25 that is fixedly secured to the distal anchor 22 at one end, slidingly passes through the proximal anchor 24, and passes proximally of the proximal anchor 24. A lock in the form of a holding clip 27 is provided to control movement of the cinch wire 25 with respect to the proximal anchor 24. The lock may be a one-way locking mechanism that permits the cinch wire 25 to be pulled proximally, but not distally, through the proximal anchor 24. Alternatively, the lock may be configured to be selectively closed by the user to hold the cinch wire 25 securely and prevent both proximal and distal movement thereof relative to the proximal anchor 24. The lock may interact with elements of the cinch wire 25, such as knots 29 or other structures on the cinch wire 25, which can enhance the holding power of the lock. Note, however, that the cinching wire may alternatively be relatively smooth and free of exterior structures such as knots, etc.

The distal anchor 22 has a distal end 30 and a proximal end 32. Similarly, the proximal anchor 24 has a distal end 34 and a proximal end 36. Both the distal anchor 22 and proximal anchor 24 have a delivery configuration and a use or deployment configuration. In the delivery configuration, the anchors 22, 24 are sized to fit into a delivery catheter for delivery into the coronary sinus. In the use configuration, the anchors 22, 24 are expanded to fit against the walls of the coronary sinus.

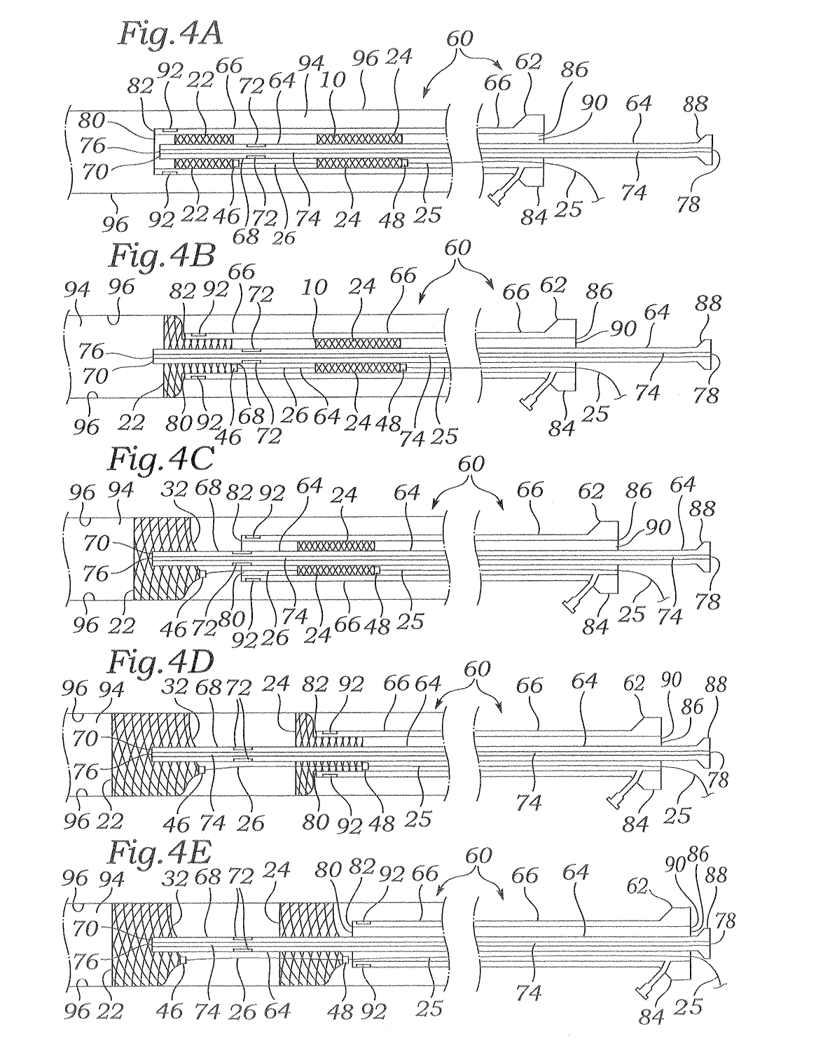

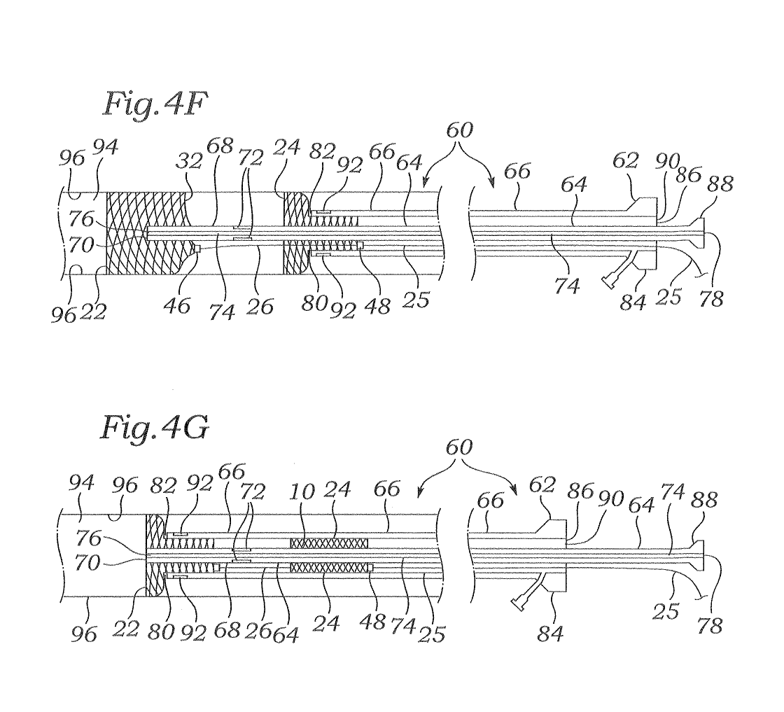

FIGS. 2A-2C depict the implant 10 with the anchors 22, 24 in their delivery configuration, wherein each anchor 22, 24 is in a retracted or collapsed condition. As depicted in FIGS. 2B and 2C, the distal anchor 22 and proximal anchor 24 each have diameters 38, 40, respectively, that during delivery are small enough to permit the anchors 22, 24 to be positioned within a delivery catheter and/or advanced through the patient's vasculature and into the coronary sinus. In the embodiment depicted, diameters 38, 40 of the anchors 22, 24 during delivery are generally equal to each other. However, depending on the particular application, the diameters 38, 40 may be different from each other during delivery. Depending on the particular application, including the type of delivery system, the diameters 38, 40, respectively, during delivery may be large enough to permit the anchors 22, 24 to be positioned around an inner rod-like element of a delivery catheter, such as that discussed in greater detail below with respect to FIGS. 4A-4E.

The distal and proximal anchors 22, 24 have lengths 42, 44, respectively. In the embodiment depicted in FIGS. 2A-3C, the distal anchor length 42 is approximately the same as the proximal anchor length 44. However, depending on the particular application, the anchor lengths 42, 44 may be different between the two anchors 22, 24. For example, because the proximal anchor of an implant is generally deployed in a larger portion of the coronary sinus, the proximal anchor of a particular implant is often larger and may preferably be longer than the distal anchor.

In FIGS. 3A-3C the anchors 22, 24 are expanded to their use or deployment configuration, wherein the distal and proximal anchors 22, 24 are expanded to their use configuration wherein the diameters 38, 40, respectively, are enlarged. The diameters 38, 40 of the anchor 22, 24 in the use configuration may be sized to fit within a selected section of the coronary sinus in which the particular anchor is to be deployed. The particular use diameter of a particular anchor may be the same size as the diameter of the selected deployment section of coronary sinus, or the use diameter may be slightly larger than the diameter of the selected deployment section of coronary sinus in order for the implant to press against the coronary sinus wall. In the particular embodiment depicted, the diameter 38 of the distal anchor 22 when expanded is smaller than the diameter 40 of the proximal anchor 24 when expanded. The larger diameter 40 of the expanded proximal anchor 24 permits the proximal anchor 24 to be deployed within the somewhat larger portion of the coronary sinus adjacent the coronary ostium.

One or both of the anchors 22, 24 may be self-expanding and biased toward the deployed configuration. The anchors 22, 24 may be formed from a shape memory metal such as Nitinol, or from other materials such as stainless steel, other metals, plastic, etc. The materials of the anchors 22, 24 and bridge portion 26 are preferably biocompatible. As an example of braided metal anchors, one or more wires of 0.0005 inches to 0.020 inches diameter could be formed into braided anchors having a braid density small enough to prevent thrombosis. The specific number of wires to form an anchor depends on the particular application, with 16 to 132 wires being a range of wires that are well within the scope of the invention.

The anchors 22, 24 and/or bridge 26 may include one or more visualization references. The embodiment of FIGS. 2A and 3A includes visualization references in the form of radiopaque marker bands 46, 48 positioned adjacent the proximal ends 32, 36 of the distal and proximal anchors 22, 24, respectively. The radiopaque marker bands 46, 48 are viewable under a fluoroscope, so that a surgeon or other user can use a fluoroscope to visualize the position of the anchors 22, 24 within the patient and with respect to any delivery catheter or other delivery devices present, such as guidewires, etc. Depending on the particular application, the visualization markers on a particular implant, such as the radiopaque marker bands 46, 48 of FIGS. 2A and 3A, may be identical or may be different from each other. Radiopaque marker bands or other visualization references that provide different radiopaque or other visualization signatures permit a user to differentiate between particular elements of a particular implant. For example, in the embodiment of FIGS. 2A and 3A, different radiopaque signatures from the distal anchor marker band 46 and the proximal anchor marker band 48 would permit the user to distinguish between the distal anchor 22 and proximal anchor 24, and thus better visualize the location and orientation of the implant 10, when viewing the implant 10 in a patient's body under fluoroscopy.

In the embodiment of FIGS. 3A-3C, the anchors 22, 24 in their expanded or deployed configuration include generally open lumens 50, 52, respectively, passing axially therethrough. These generally open lumens 50, 52 permit fluid, such as blood, to flow relatively freely through the expanded anchors 22, 24 and thus reduce the likelihood of occlusion. The marker bands 46, 48 are positioned at the edges of the anchors 22, 24 in order to preserve the inner lumen opening. As can be better seen in FIGS. 3B and 3C, substantially the entire structure of the anchors 22, 24 is positioned at or adjacent the periphery of the anchors 22, 24. It can thus be seen that when the anchors 22, 24 are expanded to deploy in a body lumen, substantially the entire structure of the anchors 22, 24 would be at or adjacent the body lumen wall(s), thereby leaving the body lumen generally unobstructed so that blood can flow freely through the relatively large generally open lumens 50, 52.

In the particular embodiment depicted, the bridge 26 is fixedly secured to the proximal end of the distal anchor 22, but slidingly passes (in the form of the cinching wire 25) through the proximal anchor 24 via a cinch wire lumen 54. In the particular embodiment depicted, the cinch wire lumen 54 is laterally offset with respect to the proximal anchor central lumen 52, and passes through the marker band 48 of the proximal anchor 24.

In the particular embodiment of FIGS. 2A-3C, the lumens 50, 52 could serve as guidewire lumens. Alternatively, a dedicated guidewire lumen or lumens could be provided, which could pass through one or both of the anchors 22, 24 and/or through the bridge 26. Dedicated guidewire lumens may preferably be laterally offset with respect to the anchors 22, 24, and could pass through the marker bands 46, 48 of the anchors 22, 24 in similar fashion to the manner in which the cinch wire lumen 54 of FIGS. 2A and 2C passes through the proximal anchor marker band 48. Whether one or more dedicated guidewire lumens are present in a particular implant, and the specific configuration of the guidewire lumen or lumens, depends on the particular application, including factors such as whether the delivery system includes a separate guidewire lumen, the type of delivery catheter (over-the-wire, rapid exchange, etc.), and the preference of the user.

As depicted in FIGS. 2B and 3B, the anchors 22, 24 have proximal ends 32, 36 that are configured to be retracted into a sheath and/or to have the sheath advanced over the proximal ends 32, 36 and anchors 22, 24. In the particular embodiment of FIGS. 2B and 3B, the anchor proximal ends 32, 36 are tapered in a generally wedge shaped to permit their withdrawal into a sheath, and/or to permit a sheath to be advanced over the anchors 22, 24. The anchors 22, 24 also include proximally trailing structures, which in the embodiment depicted are the radiopaque marker bands 46, 48 and the cinching wire 25. These proximally trailing structures can serve as grasping sites by which a user can grasp one or more of the anchors during deployment and/or retrieval of the anchors and/or implant.

FIGS. 4A-4E depicts a schematic of an implant delivery system 60 with delivery catheter 62 and implant 10 according to an embodiment of the current invention. Note that the dimensions depicted in FIGS. 4A-4E are not to scale: For example, the distance between the distal anchor 22 and the proximal anchor 24, and/or of the corresponding delivery catheter portions, in most embodiments would be substantially longer. Also, an actual delivery catheter 62 would, in most embodiments, be flexible and would have a generally curved configuration to match the curves of the particular body lumen involved.

In the pre-implant-deployment condition of FIG. 4A, the delivery system 60 includes an implant 10 and a delivery catheter 62. The delivery catheter 62 includes an inner member 64 passing within an outer sheath 66. The implant 10 is positioned on a distal portion 68 of the inner member 64, with the distal anchor 22 at or adjacent the distal end 70 of the inner member 64. The bridge 26 is positioned along the inner member 64 proximally of the distal anchor 22, and the proximal anchor 24 is positioned proximally of the both the bridge 26 and the distal anchor 22. The inner member 64 may include one or more visualization references, such as an inner member fluoroscopic marker band 72. The inner member 64 may include an inner member lumen 74 passing therethrough, which may be used as a guidewire lumen and/or other uses, depending on the particular application including factors such as whether the implant 10 itself includes a guidewire lumen therethrough. The inner member lumen 74 terminates in an inner member distal opening 76 and an inner member proximal opening 78. An inner member lumen 74 that is used as a guide wire lumen may have a diameter appropriate for the guidewire(s) to be used in the implantation procedure. For example, if a 0.035 inch diameter guidewire were used in an implantation procedure, the delivery catheter 60 could be configured with an inner member lumen 74 having in inner diameter of 0.040 inches or more. The delivery catheter 62 could be a so-called over-the-wire system or a rapid-exchange system, depending on the particular application.

In the predeployment configuration depicted in FIG. 4A, the inner member 64 and implant 10 are positioned within the outer sheath 66, with the implant distal anchor 22 and inner member distal end 70 positioned at or adjacent a distal opening 80 of the outer sheath 66. The sheath distal opening sheath 80 is surrounded by a leading edge 82 of the outer sheath 66. The delivery catheter 60 has a proximal end 84 with an outer sheath proximal opening 86 out of which the inner member proximal end 88 extends. A hemostasis valve 90 may be positioned at the outer sheath proximal opening 86 in order to prevent blood or other fluid from leaking out of the outer sheath proximal opening 86, but which also allows the inner member 64 to be advanced into and/or retracted from the outer sheath proximal opening 86. A visualization element, such as a sheath distal opening radiopaque marker band 92, may be provided on the sheath 66 at or adjacent the sheath distal opening sheath 80.

The implant 10 may be provided pre-loaded onto the delivery catheter 62, or may be loaded thereon by the user. One method for loading the implant 10 onto the delivery catheter 62 (either by the user or at the point of manufacture) involves collapsing the anchors 22, 24 into their delivery state and positioning the outer sheath 66 around the anchors 22, 24 to retain them in their collapsed/delivery state. First, the anchors 22, 24 are positioned around the inner member 64, with the distal anchor 24 adjacent a distal end 70 of the inner member 64, and the bridge 26 and proximal anchor 24 positioned proximally of the distal anchor 22. The proximal anchor 24 is collapsed and the outer sheath 66 is distally advanced over the proximal anchor 24 until the outer sheath 66 covers the proximal anchor 24. The outer sheath 66 is further advanced until the bridge 26 is covered by the outer sheath 66. Finally, the distal anchor 22 is collapsed and the outer sheath 66 is slidingly advanced over the distal anchor 22. Depending on the particular application, the outer sheath distal opening 80 may be positioned just adjacent the inner member distal end 70. The outer sheath distal opening 80 may be sealed to prevent unwanted fluid from entering the sheath 66. For example, a relatively tight silicone sleeve (not shown) could be provided that seals the outer sheath distal opening 80 to the inner member 64 while permitting the inner member 64 to be advanced out of the outer sheath distal opening 80. In the particular embodiment of FIG. 4A, the cinching wire 25 trails through the outer sheath 66 and exits out of the outer sheath proximal opening 86, thus permitting a user to grasp and pull the cinching wire 25 as desired.

In a procedure to deploy the implant 10 within a patient's body, the delivery catheter 62, with implant 10 positioned therein, is first advanced into a patient's vasculature, typically by advancing the delivery catheter over a guidewire that leads into the patient's vasculature to the desired deployment site. The delivery catheter 62 is advanced until the implant 10 is positioned at a desired deployment site in a body lumen 94, such as a site within the coronary sinus. To deploy the implant, the distal anchor 22 is deployed first. FIG. 4B depicts the implant delivery system 60 with the external sheath 66 being slid proximally with respect to the catheter inner member 64 in order to expose the distal anchor 22. The distal anchor 22 is partially exposed by the sheath, thereby allowing a distal portion of the distal anchor 22 to expand into contact with the walls 96 of the surrounding body lumen 94.

The user can use fluoroscopy or other visualization methods to confirm placement and deployment of the anchors 22, 24 and other parts of the implant 10. As the outer sheath 66 is withdrawn from the distal anchor 22, the sheath distal opening marker band 92 will be pulled across and past the distal anchor marker 46. When the user sees (on the fluoroscope) the sheath distal opening marker band 92 move proximally of the distal anchor marker band 46, the user knows that the sheath 66 has been fully withdrawn from the distal anchor 22, and that the distal anchor 22 should now be fully deployed as depicted in FIG. 4C. Note that when the distal anchor 22 of the particular embodiment depicted is fully deployed, substantially the entire structure of the distal anchor 22 is positioned against or adjacent the walls 96 of the body lumen 94, thereby leaving the body lumen 94 generally unobstructed to permit blood or other fluids to flow freely therethrough.

The user can then confirm the proper placement of the distal anchor 22 using fluoroscopy or other methods. Note that the proximal anchor 24 is still secured to the delivery catheter 62, so that the user can pull proximally on the deployed distal anchor 22 by pulling on the proximal anchor 24, which (if the cinch wire 25/bridge 26 is locked to the proximal anchor) will apply a proximal pull to the distal anchor 22 via the bridge 26. The user can also apply a proximal force on the distal anchor 22 by directly pulling on the portion of the cinch wire 25 that trails from the outer sheath proximal opening 86.

Once the distal anchor 22 is deployed, the delivery catheter 62 (and still-attached proximal anchor 24) can be pulled proximally to eliminate slack on the bridge 26 and to place the proximal anchor 24 at a desired position. The outer sheath 66 can then be further withdrawn over the inner member 68 until the outer sheath 66 begins to retract from around the proximal anchor 24, as depicted in FIG. 4D. Before or during this retraction from the proximal anchor, the user can confirm (via fluoroscopy or other methods) that the (non-deployed) proximal anchor 24 is at a desired deployment position within the coronary sinus or other body lumen. As the outer sheath 66 is withdrawn from the proximal anchor 24, the sheath distal opening marker band 92 will be pulled across and past the proximal anchor marker 48. When the user sees (on the fluoroscope) that the sheath distal opening marker band 92 has moved proximally of the proximal anchor marker band 48, the user knows that the sheath 66 has been fully withdrawn from the proximal anchor 24, and that the proximal anchor 24 should now be fully deployed, as depicted in FIG. 4E. The user can then confirm the proper placement of the proximal anchor 24 using fluoroscopy or other methods. Note that when the proximal anchor 24 of the particular embodiment depicted is fully deployed, substantially the entire structure of the proximal anchor 24 is positioned against or adjacent the walls 96 of the body lumen 94, thereby leaving the body lumen 94 generally unobstructed to permit blood or other fluids to flow freely therethrough.

After both the distal and proximal anchors 22, 24 have been deployed, the delivery catheter 62 and inner member 64 may be kept in place in the body lumen 94, such as the coronary sinus, long enough for the anchors 22, 24 to completely expand and for their proper positioning to be confirmed. The delivery catheter 62 can also be kept in, or re-advanced into, the coronary sinus to reposition one or more of the anchors 22, 24. Even after both anchors 22, 24 have been deployed, one or both of the anchors 22, 24 can be retrieved and/or repositioned. The delivery catheter 62 (or another device such as a catheter specifically configured for implant retrieval) can be placed adjacent the proximal anchor 24, with the outer sheath leading edge 82 engaging the generally-wedge-shaped proximal end 36 of the proximal anchor 24 to thereby cause the proximal anchor to collapse, as depicted in FIG. 4F. The user can simultaneously apply a holding or proximal force to the proximal anchor 24 to prevent it from distally advancing during retrieval. In an embodiment such as that depicted in FIG. 4F wherein the device 10 has a trailing proximal portion of the cinch wire 25, the proximal force can be applied via proximal pulling on the cinch wire 25 as the outer sheath 66 is advanced against and over the proximal anchor 24. As the proximal anchor 24 collapses, the sheath 66 is advanced distally over the proximal anchor 24, and/or the proximal anchor 24 is pulled proximally into the sheath 66, thereby moving the proximal anchor 24 with respect to and into the sheath 66. Once the proximal anchor 24 is completely collapsed and positioned within the outer sheath 66, the proximal anchor 24 can be repositioned and redeployed to a new desired location.

The distal anchor 22 could also be retrieved for repositioning and/or removal, as depicted in FIG. 4G. With the proximal anchor 24 collapsed within the outer sheath 66 (either after the proximal anchor is retrieved or before its initial deployment), distal anchor can be retrieved by positioning the delivery catheter 62 adjacent the distal anchor 22, which can include positioning the inner member 64 within the distal anchor 22. The outer sheath 66 can then be advanced so that the outer sheath leading edge 82 engages against the generally wedge-shaped proximal end 32 of the distal anchor 22, thereby causing the distal anchor 22 to collapse and permitting the outer sheath 66 to be advanced over the collapsed distal anchor 22. The distal anchor 22 may be pulled proximally and/or held in position to prevent its distal advancement in response to the distal pressure from the distal advancement of the outer sheath 66. The distal anchor 22 may be held in position merely by the pressure from the coronary sinus against the distal anchor 22. A holding and/or proximal force can also be applied to the distal anchor 22 by the user during anchor retrieval. For example, the delivery catheter can apply a proximal and/or holding force to the (deployed) distal anchor 22 via the bridge 26/cinch wire 25 (and/or other attached element(s) that may still be attached to the inner member and/or other portions of the delivery catheter). As the outer sheath 66 advances back over the distal anchor 22, the leading edge 82 of the outer sheath 66 will press against the tapered wedge-shaped proximal end 32 of the distal anchor 22, thereby causing the distal anchor 22 to collapse. Once the distal anchor 22 is collapsed and positioned with the outer sheath 66, the user can reposition and redeploy the distal anchor 22 at the desired location as discussed above, or remove the implant 10 and delivery catheter 62 completely. The process of anchor deployment, anchor position confirmation, anchor retrieval, and anchor redeployment can be repeated for each of the distal and proximal anchors until both anchors are at the desired location.

With both anchors 22, 24 retrieved and collapsed within the outer sheath 66, the user can redeploy the anchors 22, 24 at desired locations in the patient's body and then withdraw the delivery catheter 62, leaving the implant 10 deployed in the coronary sinus or other desired location. Alternatively, the user can leave the retrieved anchors 22, 24 within the delivery catheter outer sheath 66, and then remove the delivery catheter 62 and the implant 10 entirely from the patient's body.

Variations from the above-described embodiment are within the scope of the invention. For example, although the embodiment depicted in FIGS. 2B and 3B permit both the distal anchor 22 and proximal anchor 24 to be contracted into a delivery sheath after deployment, an implant within the scope of the invention might include anchors where only the distal anchor is easily retracted for redeployment, and/or where only the proximal anchor is easily retracted for redeployment.

Other delivery systems and methods are also within the scope of the invention. For example, the delivery catheter (or a dedicated retrieval catheter) could include a grasper configured to selectively grasp the cinch wire, one or both anchor proximal portions, or some other structure in order to apply a proximal pull to one or both anchors during anchor retrieval and outer sheath distal advancement. The delivery system may include additional features such as a dilator element (which may comprise a separate catheter) and/or a guide catheter to enhance the approach of the delivery catheter into the coronary sinus. A delivery system and method that can be used within the scope of the current invention is described in U.S. patent application Ser. No. 10/979,838, filed Nov. 1, 2004, the entire disclosure of which is incorporated herein by reference.

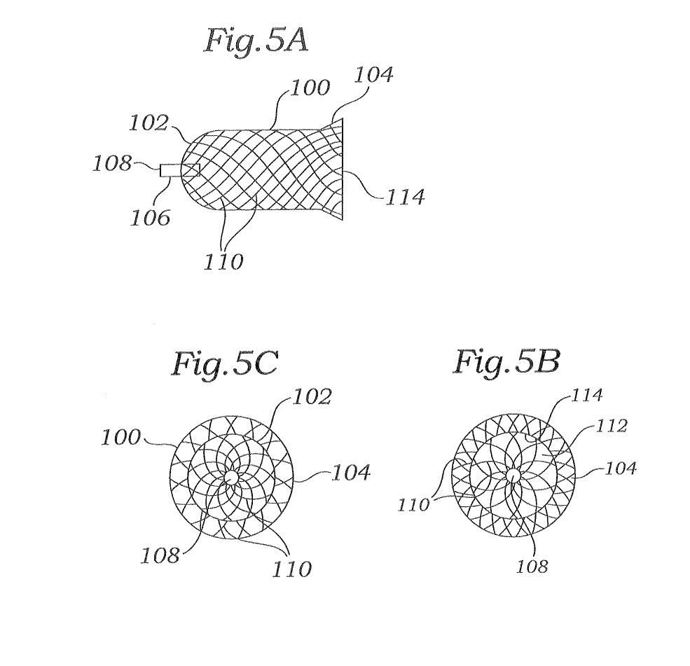

Alternate embodiments of anchors according to the invention are depicted in FIGS. 5-8. FIG. 5A-5C depict an anchor 100 in its use/deployed state and having a proximal end 102 that is substantially dome-shaped. The proximal end 102 tapers in a generally dome-shaped configuration to facilitate retrieval of the anchor 100 using a catheter having an outer sheath such as that depicted in FIGS. 4A-4E. The dome-shaped proximal end 102 permits a sheath to be advanced over the anchor proximal end 102, and/or retraction of the anchor proximal end 102 into a sheath, to permit the anchor 100 to be retrieved.

The embodiment of FIGS. 5A-5C further includes a flared distal end 104 in the expanded/use state. The flared distal end 104 provides the anchor 100 with additional anchoring capability within the coronary sinus. The anchor 100 includes a visualization element in the form of a radiopaque marker band 106 adjacent the dome-shaped proximal end 102, and a guide wire lumen 108 passing through the radiopaque marker 106 and dome-shaped proximal end 102. The anchor 100 is formed from a plurality of wires 110 formed into a wire mesh surrounding an inner lumen 112. The anchor 100 could be formed from a self-expanding material such as memory metal, including nitinol, etc. One skilled in the art will appreciate that anchor 100 depicted in FIGS. 5A-5C could be used as a distal and/or proximal anchor for an implant, and also that the dome-shaped proximal end 102 could be used without the flared distal end 104, and vice versa. For example, the flared distal end 102 of FIGS. 5A-5C could be used in combination with the generally wedge-shaped proximal ends of the expanded anchors depicted in FIGS. 3A-3C.

In the embodiment depicted in FIGS. 5A-5C, the flared distal end 104 has a relatively large and open distal opening 114 over which there is no wire mesh (although a wire mesh covering could be provided over the flared distal end 104 depending on the particular application). In contrast to the open flared distal end 104, the dome-like distal end has a wire mesh forming an interlacing structure that creates a screen-like structure over essentially the entirety of the proximal end 102, and the radiopaque marker band 106 and guide wire lumen 108 are generally centered along the axis of the anchor 100. To provide improved blood and/or other fluid flow through the anchor 100, the distal end and/or proximal end could alternatively be configured with relatively large openings therein. For example, the proximal end 102 of the anchor 100 could be configured with a relatively open proximal end having only a partial dome-like structure and having the radiopaque marker band 106 and guide wire lumen 108 axially offset towards the side of the anchor 100, similar to the relatively open proximal ends of the anchors 22, 24 depicted in FIGS. 3A-3C, and thereby providing a relatively large proximal opening into the anchor inner lumen 114 so that blood and/or other fluids could flow freely through the anchor 100 when deployed. In such an embodiment, the anchor inner lumen 114 would extend through the length of the anchor 100.

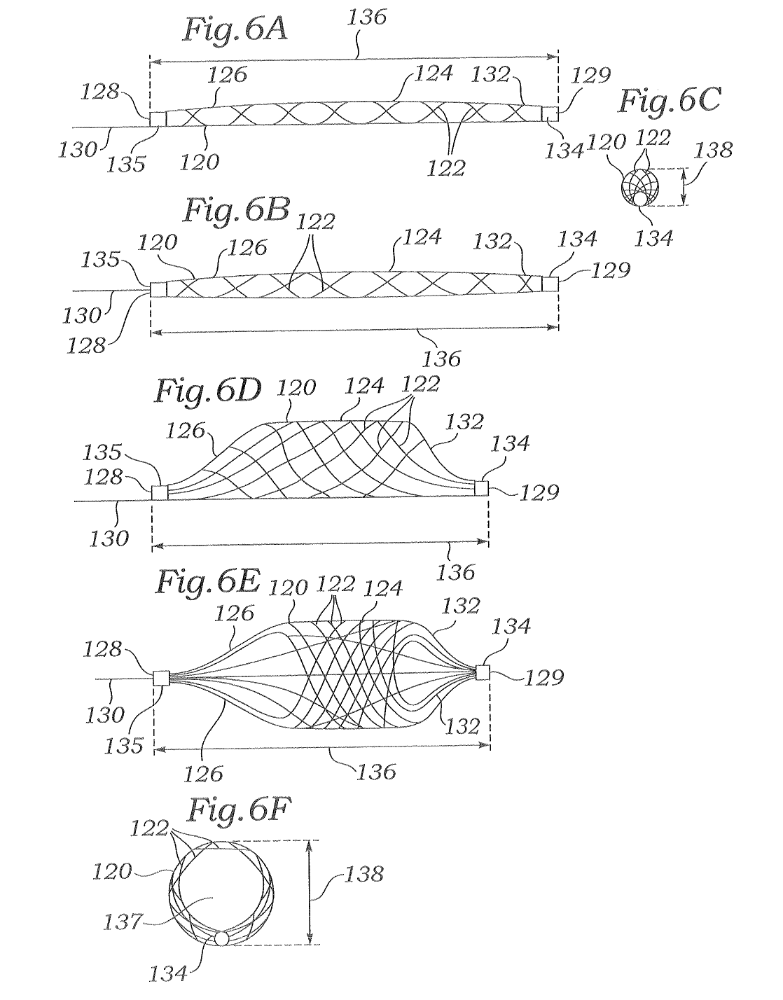

Yet another exemplary embodiment of a distal anchor 120 is shown in FIGS. 6A-6F. The distal anchor 120 is shaped by a plurality of wires 122 and includes an anchor body 124, a transition section 126 located adjacent a proximal end 128 of the distal anchor 120 and adjacent a bridge 130, and a distal end section 132 having a distal connector 134 at the anchor distal end 129 to which the plurality of wires 122 are connected. The plurality of wires 122 may be held together at the proximal transition section 126 via a proximal connector 135. Visualization references may be present, such as radiopaque markers that may be part of, or adjacent to, one or both of the distal and proximal connectors 134, 135, respectively. The plurality of wires 122 of the distal anchor 120 are wrapped into a bundled configuration in the transition (e.g., proximal) section 126 and in the distal end section 132 such that the transition and distal sections 126, 132 maintain an organized structure even as the anchor body 124 changes from the compressed/delivery state to the expanded/use state.

In the delivery state depicted in FIGS. 6A-6C, the distal anchor 120 is radially contracted to be received within a delivery catheter, and the transition section 126, the anchor body 124, and the distal end section 132 all have substantially the same diameter. Additionally, the distal anchor 120 has a length 136 which, during delivery in one exemplary embodiment, is between about 5 mm and 30 mm. In the expanded/use state depicted in FIG. 6D-6F, the anchor body 124 has a diameter 138 when expanded that is substantially larger than it is during the delivery state. In one exemplary embodiment, the diameter 138 when the anchor is expanded is about equal to or slightly larger than the diameter of an unexpanded coronary sinus. More specifically, the diameter 138 of the anchor body 124 in the use state is between about 2 mm and about 20 mm. Note that the transition section 126 and distal end section (with connector 134) are offset to one side of the expanded distal anchor 120, and the wires 122 are formed in a pattern that forces the wires to the outer periphery of the anchor 120, even at the transition section 126 and distal end section 132. Thus, when the distal anchor 120 is deployed in a body lumen, substantially the entire structure of the distal anchor 120 will be positioned against or adjacent the walls of the body lumen. The result is a relatively large unobstructed central lumen 137 through the distal anchor 120 in its deployed state that permits blood and/or other fluids to flow freely therethrough, so that the body lumen is relatively unobstructed by the anchor 120.

The distal anchor 120 shortens appreciably when it expands to its use/deployed state. In the use/deployed state, the length 136 is significantly smaller than when the distal anchor 120 is in its delivery configuration. In one exemplary embodiment, the length 136 during use/deployment is between about 5 mm and 200 mm.

The distal anchor 120 may be adapted to be transformable between the use state and the delivery state by the application of a force at a single point. More specifically, the distal anchor 120 may transformable from the use state to the delivery state by applying a single point proximal force to the transition section 126 (e.g., by pulling the transition section proximally using a retraction device). The ability to use a single point force to change the state of the distal anchor 120 allows the distal anchor 120 to be retracted from within the coronary sinus and relocated using a delivery system configured to apply the single point force to the distal anchor 120.

It will be understood by those skilled in the art that a proximal anchor (not shown) having substantially the same structure as the distal anchor 120 of FIGS. 6A-6F may be placed at an opposite end of a bridge from the distal anchor 120 as a proximal anchor to provide a complete coronary sinus retracting implant. Additionally, the proximal and distal anchors may include radiopaque marker bands on both the transition and distal end sections to allow the anchors to be better located, visualized, and/or distinguished under fluoroscopy.

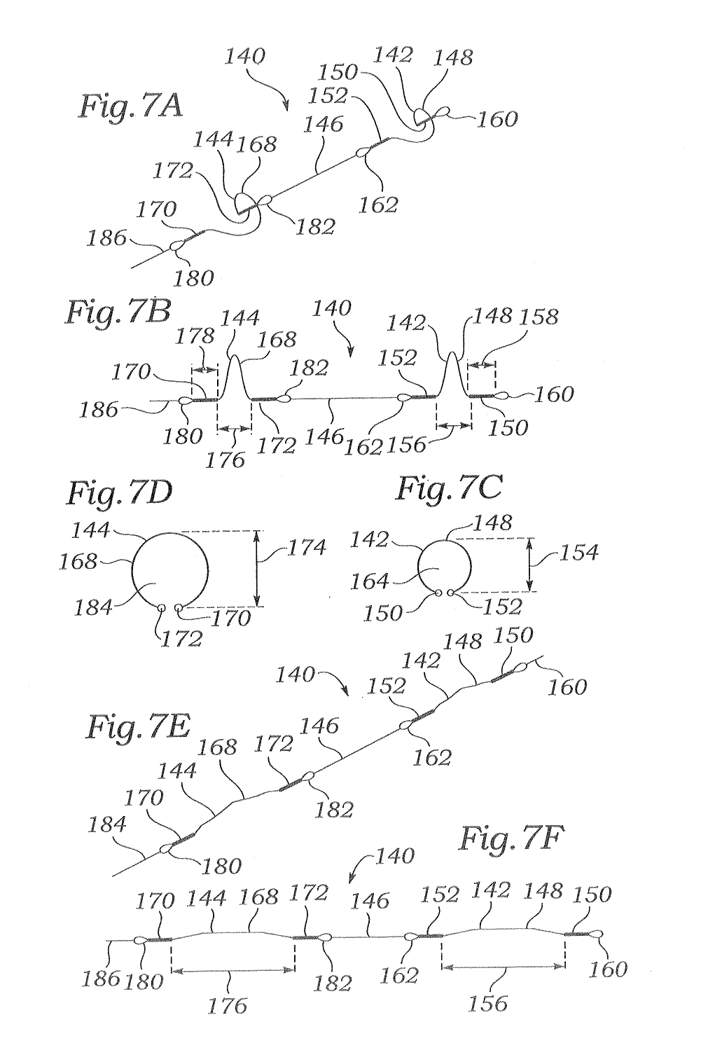

Referring now to FIGS. 7A-7E, another exemplary embodiment of a mitral valve repair implant 140 of the present invention is shown. The implant 140 includes a distal anchor 142, a proximal anchor 144, and a bridge 146 located between the distal anchor 142 and the proximal anchor 144. The anchors 142, 144 are each made of a wire formed into a loop structure, and the loops are collapsible when constrained and expand when released. The implant 140 may be made from a single piece of material, such a single wire that forms the bridge and both anchors, or may be made from separate pieces of material and subsequently joined together by, for instance, welding.

The distal anchor 142, which is depicted in proximal end view in FIG. 7C, includes a distal anchor helix coil 148, a distal anchor distal support section 150 located distally adjacent to the distal anchor helix coil 148, and a distal anchor proximal transition section 152 located proximally adjacent to the distal anchor helix coil 148.

The distal anchor helix coil 148 has a generally circular cross-sectional configuration that when expanded has a diameter 154 adapted to be about equal to or slightly larger than a diameter of a distal region of the coronary sinus in which the distal anchor 142 will be deployed. For many coronary sinus applications, the distal anchor helix coil 148 has an expanded diameter between about 1 mm and 10 mm, and (depending on the particular application) more preferably an expanded diameter between about 4 mm and 8 mm. Additionally, the distal anchor helix coil 148 has a distal anchor coil length 156 created by the spiral nature of the distal anchor helix coil 148, the distal anchor coil length 156 being measured generally as the distance between ends of the distal anchor helix coil 148. The distal anchor coil length 156 is sufficient to securely anchor the distal anchor 142 in the coronary sinus when the distal anchor 142 is expanded. In one exemplary embodiment, the distal anchor coil length 156 when the distal anchor 142 is expanded is between about 1 mm and about 20 mm, and (depending on the particular application) more preferably between about 4 mm and 8 mm.

The distal anchor distal support section 150 extends distally from the distal anchor helix coil 148 and serves as an additional anchoring support for the distal anchor 142. The particular length 158 of the distal anchor distal support section 150 may vary depending on the size of the particular distal anchor 142 and the amount of anchoring support needed. For example, the length 158 of the distal anchor distal support section 150 may be between about 1 mm and 10 mm, and (depending on the particular embodiment) more preferably between 4 mm and 6 mm. In one embodiment, the distal anchor distal support structure 150 terminates in a loop 160 to ensure that the distal anchor 142 has an atraumatic end.

The distal anchor proximal transition section 152 extends proximally from the distal anchor helix coil 148 and serves as an attachment point for the bridge 146. For example, where the distal anchor 152 is formed from a different piece of material than the bridge 146, the distal anchor proximal transition section 152 may include a loop 162 or other structure to which a bridge 146, which may be in the form of a wire or other filament, can be attached. When the implant 140 is made from a single piece of material, the distal anchor proximal transition section 152 serves as a spacer between the distal anchor helix coil 148 and the bridge 146. Depending on the particular embodiment, the distal anchor proximal transition section may also serve to help keep the implant 140 relatively straight and maintain the distal anchor 142 in a proper position during delivery and/or deployment.

The distal anchor 142 may include at least one visualization reference, such as a radiopaque marker band that serves to allow the distal anchor 142 to be located under fluoroscopy. For example, the distal anchor 142 may have a distal radiopaque marker band (not shown) located distally adjacent to the distal anchor helix coil (e.g., on the distal support section), and/or a proximal radiopaque marker band (not shown) located proximally adjacent to the distal anchor helix coil (e.g., on the proximal transition section).

When the distal anchor 142 is expanded to deploy in a body lumen, the distal anchor helix coil 148 will pass along and engage against the walls of the body lumen. The distal anchor distal support section 150 and the distal anchor proximal transition section 152, as well as the loops 160, 162 and any visualization references, will also be positioned against or adjacent the walls of the body lumen, thereby leaving a relatively large unobstructed lumen-like opening 164 through the expanded distal anchor 142 as depicted in FIG. 7C. The body lumen will thus be generally unobstructed by the deployed distal anchor, and blood and/or other fluid may flow freely therethrough.

As shown in greater detail in FIG. 7D, the proximal anchor 144 includes a proximal anchor helix coil 168, a proximal anchor proximal support section 170 located proximally adjacent to the proximal anchor helix coil 168, and a proximal anchor distal transition section 172 located distally adjacent to the proximal anchor helix coil 168. The proximal anchor helix coil 168 has a generally circular cross-sectional configuration having a proximal helix coil diameter 174 when expanded adapted to be about equal to or slightly larger than a diameter of a region of the coronary sinus adjacent the coronary ostium, or (depending on the particular application) the diameter of the particular lumen in which the proximal anchor 144 is to be deployed. More specifically, the proximal anchor helix coil 168 when expanded in situ has a diameter 174 of between about 1 mm and 30 mm, and (depending on the particular application) more preferably a diameter between about 7 mm and 25 mm. Additionally, the proximal anchor helix coil 168 has a proximal anchor coil length 176 created by the spiral nature of the proximal anchor helix coil 168, the proximal anchor coil length 176 being measured generally as the distance between ends of the proximal anchor helix coil 168. The proximal anchor coil length 176 is sufficient to securely anchor the proximal anchor 144 in the coronary sinus. The proximal anchor coil may have a length and/or diameter when deployed that is different from (e.g., larger or smaller) than the corresponding dimensions of the distal anchor coil, or the dimensions may be essentially the same between the distal and proximal anchors. In one exemplary embodiment, proximal anchor coil length 176 is between about 1 mm and about 25 mm, and more preferably between about 4 mm and 8 mm.

The proximal anchor proximal support section 170 extends proximally from the proximal anchor helix coil 168 and serves as an additional anchoring means for the proximal anchor 144. The particular length 178 of the proximal anchor proximal support section 170 may vary depending on the size of the proximal anchor 144 and the amount of support needed. For example, in one exemplary embodiment the proximal support section length 178 is between about 1 mm and about 10 mm, and more preferably between about 4 mm and 6 mm. In one exemplary embodiment, the proximal anchor proximal support section 170 includes a loop 180 at a proximal end to ensure that the proximal anchor 144 has an atraumatic end, and may also serves as an attachment point for a retrieval device to be attached, such as a retrieval line or a pair of graspers, and/or for a trailing element such as the wire 186 depicted.

The proximal anchor distal transition section 172 extends distally from the proximal anchor helix coil 168 and serves as an attachment point for the bridge 16. In the particular embodiment depicted, the proximal anchor distal transition section 172 includes a loop 182 to which the bridge 146 can be attached. When the implant 140 is made from a single piece of material, the proximal anchor distal transition section 172 serves as a spacer between the proximal helix coil 168 and the bridge 146. Depending on the particular embodiment, the proximal anchor distal transition section 172 may also serve to help keep the implant 140 relatively straight and maintain the proximal anchor 144 in a proper position during delivery and/or deployment.

The proximal anchor 144 may include at least one visualization reference, such as a radiopaque marker band that serves to allow the proximal anchor 144 to be located under fluoroscopy. For example, the proximal anchor 144 may have a distal radiopaque marker band (not shown) located distally adjacent to the proximal anchor helix coil (e.g., on the distal transition section), and/or a proximal radiopaque marker band (not shown) located proximally adjacent to the proximal anchor helix coil (e.g., on the proximal support section).

When the proximal anchor 144 is expanded to deploy in a body lumen, the proximal anchor helix coil 168 will pass along and engage against the walls of the body lumen. The proximal anchor distal transition section 172 and the proximal anchor proximal support section 170, as well as the loops 180, 182 and any visualization references, will also be positioned against or adjacent the walls of the body lumen, thereby leaving a relatively large unobstructed lumen-like opening 184 through the expanded proximal anchor 144 as depicted in FIG. 7D. The body lumen will thus be generally unobstructed by the deployed proximal anchor, and blood and/or other fluid may flow freely therethrough.

The distal anchor 142 and proximal anchor 144 of the particular embodiment of FIGS. 7A-7F may be made from biocompatible metallic wire, for example, a nitinol or a stainless steel wire. The particular wire used for the anchors 142, 144 depends on the particular application. The wire may be a round or a flat wire, and the wire may have a diameter of between about 0.001 inches and about 0.020 inches.

The distal anchor 142 and proximal anchor 144 are adapted to be transformed between a constrained/delivery configuration and an expanded/use configuration, and they may be biased toward their expanded/use configurations depicted in FIGS. 7A-7D. In the delivery configuration, the cross-sectional diameters 154, 174 of the distal and proximal anchors 142, 144 are less than the diameters in the deployed/expanded configuration. In one exemplary embodiment depicted in FIGS. 7E-7F, the anchors 142, 144 in the delivery configuration may be generally straight when in the collapsed/delivery configuration. As depicted in FIGS. 7E-7F, the anchors 142, 144 when collapsed have almost no diameter but their lengths 156, 176, respectively, are much larger than in the deployed/expanded configuration.

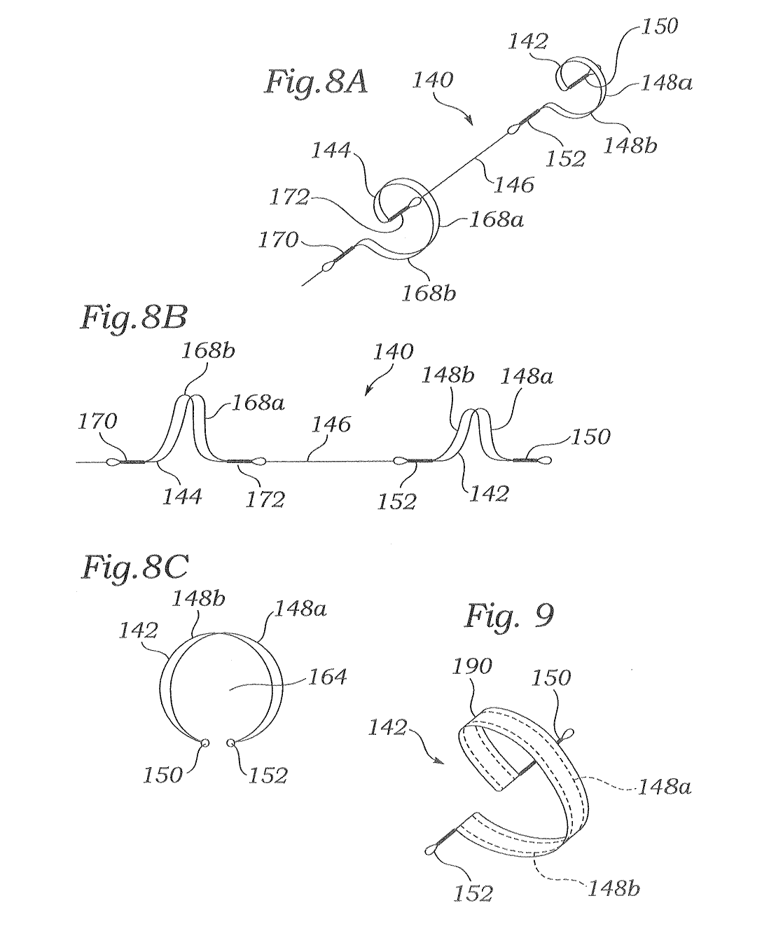

In another exemplary embodiment of the present invention as shown in FIGS. 8A-8C, the implant 140 includes a distal anchor 142 having dual distal anchor helical coils 148a, 148b, and a proximal anchor having dual proximal anchor helical coils 168a, 168b. The dual helix coils 148a-b, 168a-b of each of the anchors 142, 144 may be made from two separate wires spaced from each other. The additional helix coils provides additional anchoring ability to the anchors 142, 144. Although the embodiment shown having two helix coils 148a-b, 168a-b per anchor 142, 144, any number of coils may be used for either or both of the anchors 142, 144, and the invention is not limited to the specific embodiments described herein. Note that in the embodiment depicted, when the anchors 142, 144 are expanded to deploy within a body lumen, substantially all of the structure of the each anchor 142, 144 will be positioned against or adjacent the walls of the body lumen, thereby leaving the relatively large unobstructed lumen-like openings 164, 184 through the expanded anchors 142, 144, respectively. The body lumen will thus be generally unobstructed by the deployed proximal anchor, and blood and/or other fluid may flow freely therethrough.

One or both of the anchors may include a permanent or temporary sleeve which provides additional traction for the anchor(s). As shown in FIG. 9, in one exemplary embodiment the distal anchor 142 includes a sleeve 190 covering the dual coils 148a-b of the distal anchor 142. The sleeve 190 provides additional traction for the distal anchor 142 when the distal anchor 142 is inserted into the coronary sinus or other body lumen for deployment. Although the sleeve 190 of FIG. 9 is only depicted in the distal anchor 142, such a sleeve may be present on either one, or both, of the distal and proximal anchors 142, 144. The sleeve 190 may be permanent and may be made from a polymer, such as polyester or nylon, or it made be temporary and may be made from bioresorbable materials, such as PDS (Polydioxanon), Pronova (Poly-hexafluoropropylen-VDF), Maxon (Polyglyconat), Dexon (polyglycolic acid), and Vicryl (Polyglactin). The sleeve 190 may be attached to the particular anchor by, for example, a suture technique or by an adhesive bonding.

As depicted in FIG. 9, the sleeve 190 may cover both of the helix coils 148a, 148b together, or an individual sleeve may be used for each helix coil. For an embodiment of an anchor having more than two coils, multiple sleeves could be provided, and each individual sleeve may cover all of the coils, some of the coils, or just one of the coils. The sleeve could also be used to cover a device having one or more anchors each formed from just a single coil, such as that depicted in FIGS. 7A-F.

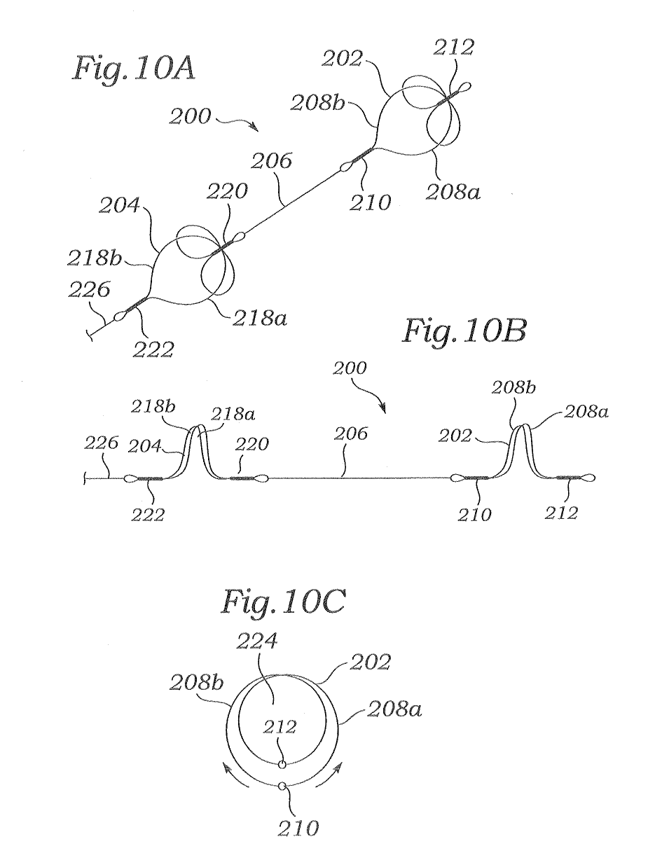

In the particular embodiment depicted in FIGS. 8A-8C, the dual helical coils 148a-b, 168a-b of each of the anchors 142, 144 pass around the periphery of the particular anchor in the same direction along the length of the implant, which in the particular embodiment depicted is a counterclockwise direction when viewed looking distally along the length of the implant. However, the dual coils of one or both anchors could be configured to pass in opposing directions to each other about the anchor periphery, as shown below with respect to FIGS. 10A-10c.

The embodiment of the present invention depicted in FIGS. 10A-C includes an implant 200 having a distal anchor 202, proximal anchor, 204, and bridge 206. The distal anchor 202 is made from two opposingly-coiled distal anchor helix coils 208a, 208b in a generally “FIG. 8” configuration. When viewed looking distally along the implant 200, the first helix coil 208a of the distal anchor 202 coils in a counterclockwise direction from a distal anchor base 210 located adjacent to the bridge 206 to a distal anchor connector 212, while the second helix coil 208b coils in a clockwise (i.e., opposite) direction from the distal anchor base 210 to the distal anchor connector 212. The first helix coil 208a and the second helix coil 208b overlap to form the generally “FIG. 8” configuration of the distal anchor 202. The first and second helix coils 208a, 208b are secured to each other at the distal anchor base 210 and/or the distal anchor connector 212 by, for example, crimping, soldering or welding. The distal anchor 202 may include a guide wire lumen, which may pass within the distal anchor base and/or the distal anchor connector. One or more visualization references, such as distal anchor radiopaque marker bands, may also be included.

FIG. 10C depicts an end view looking generally distally along the distal anchor 202. Note that the end view of FIG. 8C is partially offset from a pure end view so that the distal anchor base 210 and the distal anchor connector 212, which would be in alignment (i.e., overlapping) in a pure end view, are depicted slightly offset in order to better depict the counter-rotating distal helix coils 208a, 208b as they pass from the distal anchor base 210 to the distal anchor connector 212. When the distal anchor 202 of the embodiment depicted is expanded to deploy in a body lumen, the distal anchor helix coils 208a, 208b will pass along and engage against the walls of the body lumen. The distal anchor proximal transition section 212 and the distal anchor distal support section 210, as well as any loops and/or visualization references, will also be positioned against or adjacent the walls of the body lumen, thereby leaving a relatively large unobstructed lumen-like opening 224 through the expanded distal anchor 144 as depicted in FIG. 10C. The body lumen will thus be generally unobstructed by the deployed proximal anchor, and blood and/or other fluid may flow freely therethrough.

As depicted in FIGS. 10A and 10B, a substantially similar structure to that of the distal anchor 202 may be applied to the proximal anchor 204, with a first proximal anchor helix coil 218a and a second proximal anchor helix coil 218b coiling in opposite directions and overlapping to form the generally “figure 8” configuration of the proximal anchor 204. When viewed looking proximally along the implant 200, the first helix coil 218a of the proximal anchor 204 coils in a counterclockwise direction from a proximal anchor base 220 located adjacent to the bridge 206 to a proximal anchor connector 222, and the second proximal anchor helix coil 218b coils in a clockwise direction from the proximal anchor base 220 to the proximal anchor connector 222. The first and second proximal anchor helix coils 218a, 218b overlap to form the generally “FIG. 8” configuration of the proximal anchor 202. The first and second proximal anchor helix coils 218a,b are secured to each other at the proximal anchor base 220 and/or the proximal anchor connector 222 by, for example, crimping, soldering or welding. The proximal anchor 204 may include a guide wire lumen, which may pass within the proximal anchor base and/or the proximal anchor connector. One or more visualization references, such as proximal anchor radiopaque marker bands, may also be included.

A particular set of dual coils 208a-b, 218a-b of either of the anchors 202, 204 may be formed from a single wire, or formed from two wires which are attached by, for example, welding or an adhesive. A single wire could be used to form a portion of each of the anchors and/or the bridge. For example, a first wire could form the first distal anchor coil 208a, then coil around or along the bridge 206, then form the first proximal anchor coil 218a. The first wire, or a second wire configured for the purpose, could then loop back to form the second proximal anchor coil 218b, coil around or along the bridge 206, then form the second distal anchor coil 208b. Similarly to previously described embodiments, a cinching wire 226 may be attached to the proximal anchor 204 to acutely cinch the coronary sinus when the implant 200 has been deployed into the coronary sinus.

Depending on the particular embodiment, after the proximal and distal anchors are deployed, the separation distance between the anchors created by the bridge may be adjusted. The particular approach to adjusting the separation distance depends on the particular implant embodiment and application. Adjusting of the separation distance may be performed by the user and/or by inherent characteristics of the implant.

The proximal and distal anchors may be used with bridges having various structures as are generally known in the art. The bridge serves to separate the proximal and distal anchors by a certain distance and may also serve to reduce the distance between the anchors when the implant is inserted into the coronary sinus, thus allowing the implant to reduce mitral regurgitation. The bridge may be adapted to be acutely cinchable or it may be adapted for delayed release.

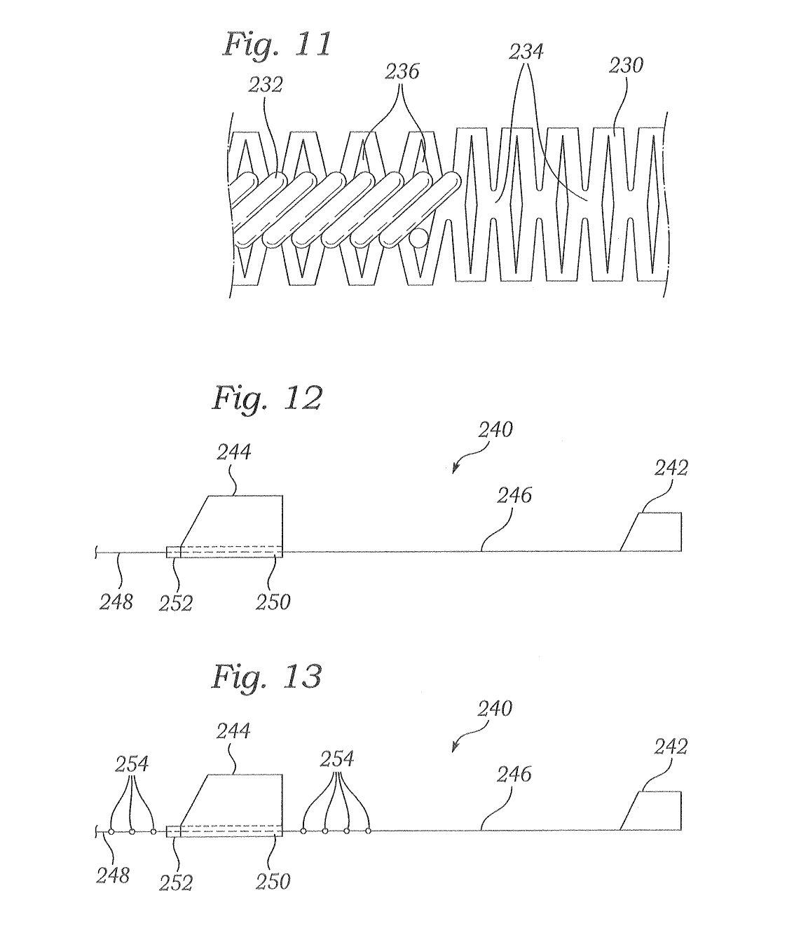

In the embodiment of FIG. 11, a section of a bridge 230 is depicted as adapted to be threaded with a resorbable material 232, which in the particular embodiment depicted is resorbable suture. Resorbable materials are those that, when implanted into a human body, are resorbed by the body by means of enzymatic degradation and/or by active absorption by blood cells and tissue cells of the human body. Examples of such resorbable materials include resorbable metals, such as magnesium alloys and zinc alloys, and resorbable polymers such as PDS (Polydioxanon), Pronova (Poly-hexafluoropropylen-VDF), Maxon (Polyglyconat), Dexon (polyglycolic acid), and Vicryl (Polyglactin). A resorbable material may be used in combination with a shape memory material, such as Nitinol, Elgiloy, or spring steel to allow the superelastic material to return to a predetermined shape over a period of time. In the particular example of FIG. 11, the bridge 232 has a spring-like shape threaded with resorbable material, and more specifically includes a section of bridge 230 with “X”-shaped bridge elements 234 and resorbable material 232 passing through openings 236 therein. The spring-like structure of the bridge 230 will contract as the resorbable material is absorbed into the body. Such an embodiment is described in pending U.S. patent application Ser. No. 11/014,273, entitled “Device for Changing the Shape of the Mitral Annulus” and filed on Dec. 15, 2004, the entire contents of which are incorporated herein.

Referring now to FIG. 12, an alternate embodiment of an implant 240 is shown. The implant includes a distal anchor 242, proximal anchor 244, and a bridge 246 adapted to provide acute cinching in the coronary sinus. More specifically, a cinching wire 248 is attached to, or forms, a proximal region of the bridge 246. The cinching wire 248 passes through a cinching wire lumen 250 in the proximal anchor 244. After the distal anchor 242 is deployed within the coronary sinus, the cinching wire 248 may be pulled proximally, thereby pulling the bridge 246 proximally and causing the distal anchor 242 to move proximally. Thus, the coronary sinus is cinched, changing the radius of curvature of the annulus of the coronary sinus. Once the coronary sinus has been cinched by a desired amount, a holding clip 252 is used to lock the cinching wire in order to maintain the bridge 246 at the proper length before the proximal anchor 244 is deployed. The proximal anchor 244 can then be deployed, and the delivery catheter removed from the patient's body. The holding clip 252 may be any device or mechanism used to hold the cinching wire 248 to the proximal anchor 244 and thereby hold the bridge 246 at a desired length. Note that the cinching or other adjustment of the length of the bridge 246 can occur prior, during, or after deployment of the proximal anchor 244, depending on the particular application.

In another alternate embodiment as shown in FIG. 13, the bridge 246 includes knots 254 which may be pulled through a holding clip 256. The holding clip 256 may adapted to allow knots 254 or similar structures on the cinching wire 248 to pass through in one direction but to prevent the knots 254 from passing back through in an opposite direction. The number of knots 254 and the spacing between the knots 254 may vary according to cinchability preferences. In this embodiment, when the bridge 246 is pulled proximally (as may occur in response to pulling on the cinching wire 248), the distance between the distal anchor 242 and the proximal anchor 244 is shortened. Bridges similar to those of FIGS. 12 and 13, as well as other bridge embodiments that can be used with the current invention, are described in pending U.S. patent application Ser. No. 11/144,521, entitled “Devices and Methods for Percutaneous Repair of the Mitral Valve via the Coronary Sinus” and filed on Jun. 3, 2005, the entire contents of which are incorporated herein.

Once the anchors are deployed, the proper placement of the implant is confirmed, and (where applicable) the bridge length is properly adjusted, the delivery catheter can be removed from the patient's body with the implant remaining inside the patient.

Various materials could be used to form the implant, delivery catheter, and other system components. For example, the inner member and/or outer sheath could be formed of braided or non-braided polymeric components. The fluoroscopic marker bands could comprise gold or other relatively highly radiopaque materials.

While the invention has been described with reference to particular embodiments, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. For example, one or more central anchors may be inserted between the proximal and distal anchors of the described implants to provide additional anchoring to the implant. Therefore, it is intended that the invention not be limited to the particular embodiments disclosed herein, but that the invention will include all embodiments falling within the scope of the appended claims.

What is claimed is:

1. An implant for placement in a coronary sinus of a patient, comprising:
a first anchor configured to be expanded from a contracted condition to an expanded condition, the first anchor having a distal end and a proximal end, the first anchor configured to radially collapse to a contracted condition in response to the application of a longitudinal force applied at a portion of the proximal end of the first anchor, the anchor configured to have a generally unobstructed central lumen passing therethrough when in the expanded configuration,
a second anchor having a distal end and a proximal end, and
a bridge having a first end and a second end, wherein the first end of the bridge is secured to the first anchor and the second end of the bridge is secured to the second anchor.

2. The implant of claim 1, wherein the proximal end of the first anchor has a generally tapered shape.

3. The implant of claim 2, wherein the proximal end of the first anchor has a generally dome-like shape.

4. The implant of claim 2, wherein the proximal end of the first anchor has a generally wedge-like shape.

5. The implant of claim 1, wherein the second anchor is a distal anchor, and the second end of the bridge is fixedly secured to the second anchor.

6. The implant of claim 1, wherein the first end of the bridge is slidingly secured to the first anchor, and the implant further comprises:
a lock configured to fixedly secure the first end of the bridge with respect to the first anchor.

7. The implant of claim 6, wherein the lock is a one-way lock that prevents sliding movement of the first end of the bridge in a first direction with respect to the first anchor, and wherein the lock permits sliding movement of the first end of the bridge in a second direction with respect to the first anchor, and wherein the second direction is opposite to the first direction.

8. An implant for placement in a coronary sinus of a patient, comprising:
a first anchor having a distal end and a proximal end, the first anchor configured to expand from a contracted condition to an expanded condition, the first anchor having a first generally helical coil and configured to radially collapse in response to the application of a longitudinal force applied at a portion of the proximal end of the first anchor, the radial coil defining an outer diameter of the first anchor in the expanded condition,
a second anchor having a distal end and a proximal end, and
a bridge having a first end and a second end, wherein the first end of the bridge is secured to the first anchor and the second end of the bridge is secured to the second anchor.

9. The implant of claim 8, wherein the first anchor comprises a self-expanding memory material.

10. The implant of claim 8, wherein the first anchor comprises a second generally helical coil.

11. The implant of claim 10, wherein the first helical coil coils in a first direction, and the second helical coil coils in a second direction, wherein the first direction is opposite to the second direction.

12. The implant of claim 8, wherein the first anchor further comprises a covering over the first helical coil.

13. The implant of claim 8, wherein second anchor has a first generally helical coil and is configured to radially collapse in response to the application of a longitudinal force applied at a portion of the proximal end of the second anchor.

14. A method of deploying an implant in a body lumen, wherein the implant includes a first anchor, a second anchor, and a bridge connecting the first anchor to the second anchor, the method comprising:
advancing the first anchor to a first anchor deployment location within a body lumen;
expanding the first anchor to an expanded condition to deploy the first anchor within a body lumen, wherein the first anchor expands into contact with the walls of the body lumen with substantially all of the first anchor structure positioned at or adjacent the walls of the body lumen to thereby leave the body lumen substantially open and unblocked;
advancing the second anchor to a second anchor deployment location within a body lumen;
expanding the second anchor to deploy the second anchor within the body lumen, wherein the second anchor expands into contact with the walls of the body lumen;
after the step of expanding the first anchor to an expanded condition, the further step of contracting the first anchor from its expanded condition to a contracted condition wherein the first anchor has a diameter significantly smaller than the diameter of the body lumen so that the first anchor can be moved proximally or distally within the body lumen.

15. The method of claim 14, further comprising:
after the step of contracting the first anchor from its expanded condition to a contracted condition, the further step of proximally moving the first anchor within the body lumen.

16. The method of claim 15, further comprising:
after the step of proximally moving the first anchor within the body lumen, the further step of re-expanding the first anchor to an expanded condition to deploy the first anchor within a body lumen, wherein the first anchor re-expands into contact with the walls of the body lumen

17. The method of claim 15, further comprising:
after the step of proximally moving the first anchor within the body lumen, the further step of removing the implant from within the body lumen.

18. The method of claim 14, wherein the first anchor is a proximal anchor and the second anchor is a distal anchor.

19. The method of claim 18, wherein the step of expanding the second anchor to deploy the second anchor within the body lumen is performed prior to the step of expanding the first anchor to an expanded condition to deploy the first anchor within a body lumen.

20. The method of claim 19, further comprising:
after the step of expanding the second anchor to deploy the second anchor within the body lumen, the further step of adjusting the length of the bridge between the first anchor and the second anchor.
